(19) United States
(12) Patent Application Publication
(10) Pub. No.: US 2024/0116887 A1
(43) Pub. Date: Apr. 11, 2024

(54) Synthesis of furandicarboxylic acid from aldaric acid

(71) Applicant: Teknologian tutkimuskeskus VTT Oy, Espoo (FI)

(72) Inventors: Nicolaas Van Strien, Espoo (FI); Sari Rautiainen, Espoo (FI); Holger Pöhler, Espoo (FI)

(21) Appl. No.: 18/272,367

(22) PCT Filed: Jan. 14, 2022
(86) PCT No.: PCT/FI2022/050025

§ 371 Date: Jul. 14, 2023

(30) Foreign Application Priority Data
Jan. 15, 2021 (FI) 20215049

(51) Int. Cl. C07D 307/68; B01J 31/02

(52) U.S. Cl. CPC C07D 307/68; B01J 31/0225

(57) ABSTRACT

According to an example aspect of the present invention, there is provided an energy efficient and environmentally benign method for producing furandicarboxylic acid (FDCA) and furandicarboxylic acid esters (FDCAE) from aldaric acid esters.

### FIELD

The present invention relates to a method for producing furandicarboxylic acid (FDCA) and furandicarboxylic acid esters (FDCAE) from aldaric acid esters.

### BACKGROUND

The shift from fossil-based polymers to renewable plastics requires new efficient methods for the production of monomers from biomass. 2,5-Furandicarboxylic acid (FDCA) and its esters (FDCAE) are promising bio-based substitutes for terephthalic acid in the production of polyesters (Bozell and Petersen, 2010; Stadler et al., 2019). Compared to fossil-based polyethylene terephthalate (PET), polyethylene furaonate (PEF) produced from FDCA has about 50% lower carbon foot print (Eerhart et al., 2012). Furthermore, PEF polymers have superior gas barrier and mechanical properties compared to PET polymers (Avantium, 2020). In addition, FDCA is rapidly gaining interest as a bio-based monomer for other applications such as polyurethanes and epoxy resins (Deng et al., 2015; Marotta et al., 2018). Furthermore, FDCA has been ranked among the 12 raw materials with the greatest industrial potential (Werpy and Peterson, 2004).

Furan carboxylates have been traditionally used for example in pharmacology, where its diethyl ester has showed a strong anesthetic activity. Furandicarboxylic acid is also a very powerful chelating agent. In medicine, it is for example used to treat kidney stones, but also in the preparation of grafts having biological properties similar to those of natural tissues, and which are characterized by a lack of rejection after transplantation.

Furan carboxylates, such as 2,5-furandicarboxylic acid, can be produced from aldaric acids. For example, WO 2016/166421 describes such method, wherein solid heterogeneous catalysts are utilized. The resultant reaction mass typically contains unreacted raw material, small amounts of side reactions and the side product furoic acid (ester) in addition to FDCA (ester). WO 2015/189481 on the other hand discloses selective catalytic dehydroxylation method of aldaric acids for producing muconic acid and furan chemicals. Drawbacks relating to these existing technologies include the use of an alcohol solvent and high amounts (50 wt-%) of solid acid catalysts.

There is a need for a novel technology, wherein the synthesis of FDCA and FDCAE is cheap, efficient and environmentally benign. Avoiding the use of expensive catalysts and solvents is thus an essential factor.

### SUMMARY OF THE INVENTION

The invention is defined by the features of the independent claims. Some specific embodiments are defined in the dependent claims.

According to an aspect of the present invention, there is provided a method for producing furandicarboxylic acid (FDCA) and furandicarboxylic acid ester (FDCAE) from aldaric acid ester.

This and other aspects, together with the advantages thereof over known solutions are achieved by the present invention, as hereinafter described and claimed.

The method of the present invention is mainly characterized by what is stated in the characterizing part of claim 1.

Considerable advantages are obtained by means of the invention. For example, the method described herein uses more soluble aldaric acid ester form than previously reported, to improve yield and reduce solvent use. In addition, a greener solvent with a lower boiling point compared to the previously used solvents is applied. Also the catalyst amount has been reduced. Otherwise, the present invention uses existing machinery and enables reuse of the raw material, which gives a benefit in raw material cost savings and increased efficiency.

Next, the present technology will be described more closely with reference to certain embodiments.

### EMBODIMENTS

The present technology provides improved and cost-efficient synthesis method of furandicarboxylic acid (ester) from aldaric acid (esters) by using bio-based non-alcoholic reaction solvent and suitable catalyst in a pressurized reactor conditions.

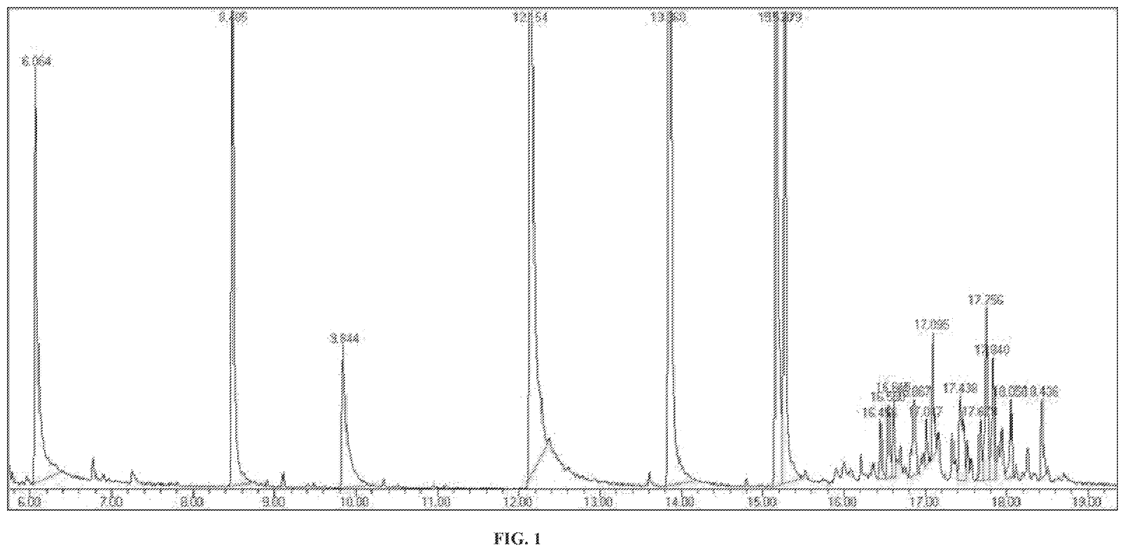

FIG. 1 is a GC-MS chromatogram showing the products formed by the present method. The visible peaks in the chromatogram are:
6 min: FCA Me ester (7.3% of the integrated peak areas)
8.4 min: FCA (6.9%)
9.8 min: unknown (3.8%)
12.1 min: FDCA di-Me ester (24.9%)
13.9 min: FDCA mono-Me ester (27.5%)
15.17 min: FDCA mono-Me ester (9.2%)
15.27 min: FDCA (6.9%)
16-19 min: silylated compounds (all unknowns together 17.3%)

FCA is abbreviation for furan carboxylic acid, FDCA for furandicarboxylic acid and FDCAE for furandicarboxylic acid ester i.e. furandicarboxylate, and are intended to cover all possible isomers thereof, such as for example 2,3- and 2,5-isomers.

According to an embodiment of the present invention, the method for producing furandicarboxylic acid (FDCA) and furandicarboxylic acid esters (FDCAE) from aldaric acid esters comprises at least the steps of:
adding an aldaric acid ester and a catalyst into a pressure reactor,
adding a bio-based non-alcoholic solvent to the reactor,
pressurizing the reactor with an inert gas to 5 bars,
increasing the temperature inside the reactor up to 240° C. and mixing the content for a pre-determined reaction time,
cooling the reactor to room temperature of 20 to 25° C.,
filtering the catalyst and removing the solvent by evaporation, and
collecting the formed product.

According to one embodiment of the present invention, the aldaric acid ester is mucic acid ester.

According to one embodiment of the present invention, the catalyst is a silica supported sulfonic acid. More precisely, it is herein preferred to use Si-Tosic acid as the catalyst. By using silica supported sulfonic acid catalysts, such as Si-Tosic acid, the amount of catalyst is drastically reduced compared to the existing technology, which uses phenylic sulfonic acid ethyl sulfide silica catalyst, which is typically 10-times more expensive.

According to one embodiment of the present invention, the solvent is acetic acid ester or formic acid ester, preferably methyl acetate. The use of methyl acetate enables the use of the methyl ester of the starting material. With the use of methyl acetate as a reaction solvent, the problems relating to formation of dimethylether when using methanol solvent (as in the existing technology), is reduced. In addition, methyl acetate has not been shown to date to be used in the synthesis of FDCA. Furthermore, methyl acetate is cheap reaction solvent that can be easily removed from the reaction mixture due to its low boiling point. It has also lower health risks compared to methanol or n-butanol.

According to one embodiment of the present invention, the reaction is carried out in a pressure reactor, such as in a Hastalloy pressure reactor. The substrate and catalyst are added to the reactor followed by solvent. The reactor is then pressurized to 5 bar with an inert gas, for example nitrogen. The temperature is increased up to 240° C., more preferably only up to 210° C., and the contents are stirred for 4 hours before cooling to room temperature. The catalyst is then filtered away and the solvent removed by evaporation. The brown-black solid isolated is crude product FDCA methyl ester.

Thus, according to one embodiment of the present invention, the reaction is carried out during 4-hour reaction time. Existing synthesis methods for FDCA typically requires at least 24-hour reactions, whereby running the reaction for 20-hours shorter saves significant amount of energy and provides improvements to the techno-economic assessment of the production process.

One further advantage of the present invention is that the FDCA synthesis route disclosed herein produces fewer side-products than previously reported. Typically, synthesis of FDCA from furfural derivatives causes multiple side-reactions, which is proving to be a major problem for industry when it comes to follow-on polymerization reactions. The synthesis of FDCA from aldaric acids produces furancarboxylic acid (ester) as a side reaction, which complicate the purification of the crude product. Having fewer side-products, as seen in FIG. 1, benefits the downstream processing.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated.

### INDUSTRIAL APPLICABILITY

At least some embodiments of the present invention find industrial application in generating a full value chain from the forest industry, agriculture, or food industry side streams to platform chemicals and end applications. In principle, this chain comprises production of aldaric acids from aldoses and side-stream carbohydrates, converting the aldaric acids to dicarboxylic acids, which in turn are used as platform chemicals for various bio-based applications, such as bio-based polyesters and nylon. According to one example, the present method produces 2,5-Furandicarboxylic acid for use in the production of polyethylene furanoate.

### EXAMPLES

Set 1: Methyl Acetate

Mucic acid methyl ester (2 g, 8.4 mmol) was added to a hastelloy C-276 pressure reactor. To this was then added Si-Tosic acid (0.095 mmol, 1.1 mol %) and methyl acetate solvent. A stirrer bar was added and the reactor was then sealed and flushed with nitrogen before pressurising to approximately 5 bar. The reactor then heated to the required temperature and stirred for a specific time. Once the reaction was completed, the reactor was cooled to room temperature and the contents removed. Vacuum filtration and evaporated of solvent (40° C., below 10 mbar) afforded the product as a solid. The reaction product was purified by using known technology and was characterized GC-MS and ¹H NMR. Yields are interpreted from GC-FID.

TABLE 1

| | | | | | | | Purified | Purified |
|---|---|---|---|---|---|---|---|---|
| | | | | | | | yield | yield |
| | | | | | | | FDCA | FDCA |
| | Reaction conditions | | | | | Purified | mono | di |
| | Catalyst | Solvent | | | Mass | yield | methyl | methyl |
| | (mol- | volume | Temperature | Time | isolated | FDCA | ester | ester |
| Entry | %) | (cm³) | (° C.) | (h) | wt-% | mol-% | mol-% | mol-% |
| 1 | 1.1 | 10 | 220 | 4 | 52 | 2.8 | 16.2 | 31.6 |
| 2 | 1.1 | 5 | 220 | 4 | 66 | 4.4 | 17.5 | 29.1 |
| 3 | 0.7 | 5 | 220 | 4 | 69 | 5.5 | 14.4 | 25.9 |
| 4 | 1.1 | 5 | 210 | 4 | 83 | 4.6 | 19.8 | 27.3 |
| 5 | 1.1 | 5 | 230 | 4 | 47 | 1.3 | 13.4 | 23.1 |
| 6 | 1.1 | 5 | 230 | 2 | 69 | 5.4 | 18.1 | 25.1 |

¹H-NMR (DMSO-d₆, 500 MHz)
FDCA dimethyl ester: δ = 7.465 (s, 2H, CH), 3.898 (s, 6H, CH₃).
FDCA monomethyl ester: δ = 7.430 (d, 1H, CH), 7.360 (d, 1H, CH), 3.889 (s, 3H, CH₃).

Set 2: Ethyl Acetate

Mucic acid methyl ester (2 g, 8.4 mmol) was added to a hastelloy C-276 pressure reactor. To this was then added Si-Tosic acid (0.095 mmol, 1.1 mol %) and ethyl acetate. A stirrer bar was added and the reactor was then sealed and flushed with nitrogen before pressurising to approximately 5 bar. The reactor then heated to the required temperature and stirred for a specific time. Once the reaction was completed, the reactor was cooled to room temperature and the contents removed. Vacuum filtration and evaporated of solvent (40° C., below 10 mbar) afforded the product as a solid. The reaction product was purified by using known technology and was characterized GC-MS and ¹H NMR. Yields are interpreted from GC-FID.

TABLE 2

| | | | | | | | Purified | |
|---|---|---|---|---|---|---|---|---|
| | | | | | | | yield | Purified |
| | | | | | | | FDCA | yield |
| | Reaction conditions | | | | | Purified | mono | FDCA |
| | | Solvent | | | Mass | yield | ethyl | di ethyl |
| | Catalyst | volume | Temperature | Time | isolated | FDCA | ester | ester |
| Entry | (mol-%) | (cm³) | (° C.) | (h) | wt-% | mol-% | mol-% | mol-% |
| 7 | 1.1 | 5 | 210 | 4 | 90 | 6.8 | 13.1 | 22.3 |

Set 3: n-Butyl Acetate

Mucic acid methyl ester (2 g, 8.4 mmol) was added to a hastelloy C-276 pressure reactor. To this was then added Si-Tosic acid (0.095 mmol, 1.1 mol %) and n-butyl acetate. A stirrer bar was added and the reactor was then sealed and flushed with nitrogen before pressurising to approximately 5 bar. The reactor then heated to the required temperature and stirred for a specific time. Once the reaction was completed, the reactor was cooled to room temperature and the contents removed. Vacuum filtration and evaporated of solvent (40° C., below 10 mbar) afforded the product as a solid. The reaction product was purified by using known technology and was characterized GC-MS and ¹H NMR. Yields are interpreted from GC-FID.

TABLE 3

| | | | | | | | Purified | |
|---|---|---|---|---|---|---|---|---|
| | | | | | | | yield | Purified |
| | | | | | | | FDCA | yield |
| | Reaction conditions | | | | | Purified | mono | FDCA |
| | | Solvent | | | Mass | yield | butyl | di butyl |
| | Catalyst | volume | Temperature | Time | isolated | FDCA | ester | ester |
| Entry | (mol-%) | (cm³) | (° C.) | (h) | wt-% | mol-% | mol-% | mol-% |
| 8 | 1.1 | 5 | 210 | 4 | 100 | 5.4 | 14.7 | 31.2 |

¹H-NMR (DMSO-d₆, 500 MHz)
FDCA dibutyl ester: δ = 7.19 (s, 2H, 2CH), 4.34 (t, 4H, 2CH2), 1.75 (m, 4H, 2CH2), 1.45 (m, 4H, 2CH2), 0.95 (t, 6H, 2CH3)
FDCA monobutyl ester: δ = 7.42 (d, 1H, CH), 7.36 (d, 1H, CH), 4.32 (t, 2H, CH2), 1.71 (m, 2H, CH2), 1.42 (m, 2H, CH2), 0.96 (t, 3H, CH3)

### CITATION LIST

### Patent Literature

WO 2016/166421
WO 2015/189481

### Non-Patent Literature

Bozell, J. J., Petersen, G. R., Green Chem., 2010, 12, 539-554.
Stadler, B. M., Wulf, C., Werner, T., Tin, S., de Vries, J. G., ACS Catal., 2019, 9, 8012-8067.
Eerhart, J. J. E., Faaij, P. C., Patel, M. K., Energy Environ. Sci., 2012, 5, 6407-6422.
Avantium YXY Technology, https://www.avantium.com/technologies/yxy/, (accessed December 2020).
Deng, J., Liu, X., Li, C., Jiang, Y., Zhu, J., RSC Adv., 2015, 5, 15930-15939.
Marotta, A., Ambrogi, V., Cerruti, P., Mija, A., RSC Adv., 2018, 8, 16330-16335.
Werpy, T., Peterson, G., Top Value Added Chemicals from Biomass, 2004, 1, 26-28.

1-8. (canceled)

9. A method for producing furandicarboxylic acid (FDCA) and furandicarboxylic acid esters (FDCAE) from aldaric acid esters, the method comprising:
adding an aldaric acid ester and a catalyst into a pressure reactor,
adding a non-alcoholic solvent to the pressure reactor,
pressurizing the pressure reactor with an inert gas,
increasing the temperature inside the pressure reactor to a temperature of up to 240° C. and mixing the contents of the pressure reactor,
cooling the pressure reactor to a temperature of 20 to 25° C.,
filtering the catalyst and removing the solvent by evaporation, and
collecting the formed product.

10. The method according to claim 9, wherein the aldaric acid ester comprises mucic acid methyl ester.

11. The method according to claim 9, wherein the catalyst comprises a silica-supported sulfonic acid.

12. The method according to claim 9, wherein the catalyst comprises Si-Tosic acid.

13. The method according to claim 9, wherein the solvent comprises an acetic acid ester or a formic acid ester.

14. The method according to claim 9, wherein the pressure reactor is pressurized with nitrogen gas.

15. The method according to claim 9, wherein the temperature inside the reactor is increased up to 210° C.

16. The method according to claim 9, wherein the mixing the contents of the pressure reactor is done for 4 hours.

17. The method according to claim 9, wherein the pressure reactor is pressurized to 5 bars.

18. The method according to claim 1, wherein the solvent is bio-based.

19. A method for producing furandicarboxylic acid (FDCA) and furandicarboxylic acid esters (FDCAE), the method comprising:
adding mucic acid methyl ester and a catalyst into a pressure reactor, wherein the catalyst comprises Si-Tosic acid,
adding a solvent comprising methyl acetate to the pressure reactor,
pressurizing the pressure reactor with an inert gas to approximately 5 bars,
increasing the temperature inside the pressure reactor to a temperature of up to 240° C. and mixing the contents of the pressure reactor,
cooling the pressure reactor to a temperature of 20 to 25° C.,
filtering the catalyst and removing the solvent by evaporation, and
collecting the formed product.
